(19) United States
(12) Patent Application Publication
(10) Pub. No.: US 2026/0290538 A1
(43) Pub. Date: Sep. 24, 2026

(54) GENERATIVE AI BASED TOOL TO PROVIDE VIRTUAL INTERACTIVE THERAPY FOR ADHERENCE

(71) Applicant: ResMed Corp., San Diego, CA (US)

(72) Inventor: Alberto EDEZA, Albuquerque, NM (US)

(21) Appl. No.: 19/572,146

(22) Filed: Mar. 19, 2026

Related U.S. Application Data
(60) Provisional application No. 63/775,190, filed on Mar. 20, 2025.

(51) Int. Cl. G16H 20/30; G16H 10/60; G16H 50/20

(52) U.S. Cl. CPC G16H 20/30; G16H 10/60; G16H 50/20

(57) ABSTRACT

A computerized system and method for providing virtual interactive therapy for enhanced adherence to a user or patient. Input data associated with a user is received and the user is categorized into a behavioral subgroup based on the input data. An initial motivational interviewing question is generated for the user based on the behavioral subgroup to which the user belongs. User response to the initial motivational interviewing question is received. A motivational interviewing session is dynamically generated for the user based on the behavioral subgroup and the user response. The motivational interviewing session includes a set of follow-up questions and support messages for the user. During the motivational interviewing session, behavior change of the user is tracked based on user responses to the set of follow-up questions and support messages. Impact of the tracked behavior change of the user on adherence to the virtual interactive therapy is quantified.

### BACKGROUND

Positive airway pressure (PAP) therapy devices, including CPAP, APAP, and BiPAP devices, are among the most widely prescribed durable medical equipment for treating sleep-disordered breathing conditions such as obstructive sleep apnea. Despite their clinical efficacy, some users stop using PAP devices due to patient discomfort, mask interface issues, and insufficient real-time responsiveness of the device to the patient's behavioral and physiological state during therapy sessions. Existing PAP device architectures collect usage telemetry, such as nightly hours of use, apnea-hypopnea index (AHI), mask leak rates, and pressure titration data, but lack integrated feedback mechanisms capable of dynamically adjusting both the therapeutic and motivational parameters of device operation based on this data. Further, existing respiratory therapy devices do not adapt to patient-specific behavioral profiles or provide motivationally-tailored communication through the device interface.

### SUMMARY

Some examples provide a computerized method for enhancing adherence of a user to a therapy using a generative artificial intelligence (AI) based virtual interactive therapy adherence tool, the method comprising: receiving input data associated with the user; categorizing the user into a behavioral subgroup based on the input data; generating a motivational interviewing question for the user based on the behavioral subgroup; receiving a user response to the motivational interviewing question; dynamically generating a motivational interviewing session for the user based on the behavioral subgroup and the user response, the motivational interviewing session including a follow-up question and support message for the user; detecting a behavior change of the user based on a user response to the follow-up question and support message during the motivational interviewing session; and measuring impact of the detected behavior change of the user on adherence to the therapy using the generative AI based virtual interactive therapy adherence tool.

Some examples provide a generative artificial intelligence (AI) based system to provide virtual interactive therapy for enhanced adherence, the system comprising: a processor; and a memory storing instructions that upon execution by the processor cause the processor to: receive input data associated with a user from a positive airway pressure (PAP) therapy device; categorize the user into a behavioral subgroup based on the input data; generate initial motivational interviewing questions for the user based on the behavioral subgroup; receive user responses to the initial motivational interviewing questions; dynamically generate a motivational interviewing session for the user based on the behavioral subgroup and the user responses, the motivational interviewing session including follow-up questions and support messages for the user; track a behavior change of the user based on user responses to the follow-up questions and support messages during the motivational interviewing session; quantify impact of the tracked behavior change of the user on adherence to the virtual interactive therapy; and based on the impact, cause adjusting one or more operational parameters of the PAP therapy device.

Some examples provide a computer storage medium storing instructions to provide virtual interactive therapy for enhanced adherence, the instructions upon execution by a processor cause the processor to: receive input data associated with a user; categorize the user into a behavioral subgroup based on the input data; generate a set of initial motivational interviewing questions for the user based on the behavioral subgroup; receive user response to the set of initial motivational interviewing questions; dynamically generate a motivational interviewing session for the user based on the behavioral subgroup and the user response, the motivational interviewing session including a set of follow-up questions and support messages for the user; track behavior change of the user based on user responses to the set of follow-up questions and support messages during the motivational interviewing session; and quantify impact of the tracked behavior change of the user on adherence to the virtual interactive therapy.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

The present description will be better understood from the following detailed description read considering the accompanying drawings, wherein:

FIG. 1 is a block diagram illustrating an example generative artificial intelligence (AI) based system to provide virtual interactive therapy for enhanced adherence;

FIG. 2 illustrates an example architecture for combining data from observable and latent variables to provide virtual interactive therapy;

FIG. 3 illustrates a block diagram of behavior change techniques, emphasizing capability, opportunity, and motivation leading to behavior change, with implementation possible at multiple levels;

FIG. 4 is a flowchart illustrating an example method for enhancing adherence of a user to a therapy using a generative artificial intelligence (AI) based virtual interactive therapy adherence tool; and

FIG. 5 illustrates an example computing apparatus as a functional block diagram.

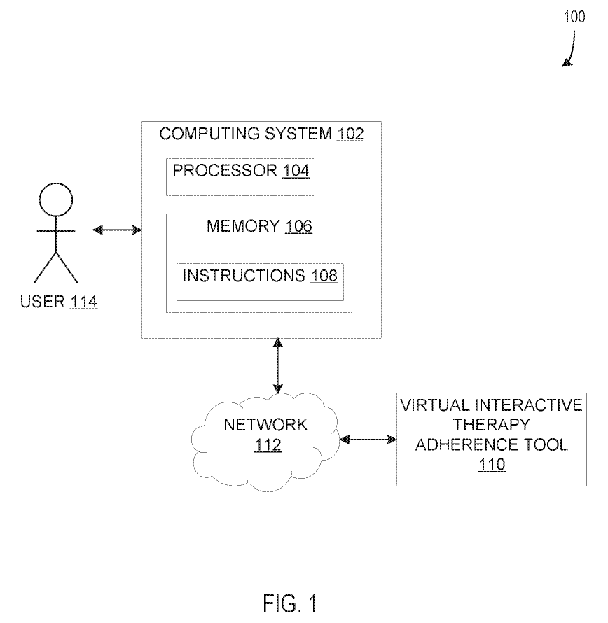

Corresponding reference characters indicate corresponding parts throughout the drawings. In FIGS. 1 to 5, the systems are illustrated as schematic drawings. The drawings may not be to scale. Any of the figures may be combined into a single example or embodiment.

### DETAILED DESCRIPTION

Adherence to medical therapies, particularly for chronic conditions, is a significant challenge in healthcare. Patients often struggle to maintain consistent use of prescribed treatments, which can lead to suboptimal health outcomes and increased healthcare costs. This issue is particularly pronounced in therapies that require long-term commitment, such as positive airway pressure (PAP) therapy for sleep apnea. Despite the proven benefits of such treatments, adherence rates remain low due to various factors, including patient motivation, understanding of the therapy, and the perceived burden of treatment.

Motivational interviewing (MI) has emerged as an effective technique to enhance patient adherence by addressing ambivalence and fostering intrinsic motivation for change. In some systems, MI is intensive and requires a minimum of 45 minutes of interaction with a trained therapist. However, availability of a trained therapist, and a level of comfort for a user to interact with the trained therapist, are example areas of concern that reduce adherence to the therapy (e.g., continuous positive airway pressure (CPAP) therapy).

Aspects of the disclosure provide a PAP therapy device and associated patient interface system that integrates onboard or cloud-coupled physiological sensors, behavioral state classification, and an adaptive interaction module capable of delivering personalized, evidence-based motivational prompts that respond to real-time therapy session data and longitudinal usage patterns.

Examples of this disclosure digitally deliver virtual interactive therapy using a generative artificial intelligence (AI) based tool that enhances adherence to the therapy. Examples of the disclosure provide a solution that supports compliance, both short term adherence (e.g., less than 90 days) and long term adherence through a clinically backed therapy solution implementing motivational interviewing via the generative AI based tool. Examples of the disclosure focus on enhancing patient engagement, improving healthcare outcomes, and streamlining the care process with generative AI technology to make this impactful support scalable. Generative AI behavioral intervention provides a cutting-edge and scalable version of the most efficacious intervention available for PAP therapy adherence. Generative AI behavioral intervention is coupled with rigorous advanced statistical analyses to examine effectiveness of the implementation.

Generative AI presents a promising avenue for replicating the nuanced and adaptive nature of MI, potentially transforming how adherence support is provided in healthcare settings. A generative AI based system as described herein provides virtual interactive therapy for adherence (VITA) for a user or patient. Input data associated with a user is received from a PAP therapy device and the user is categorized into a behavioral subgroup based on the input data. In some examples, the input data comprises one or more of: demographic information, therapy feedback from the user, device usage information, and user interactions, including the written or spoken content thereof, with a therapy adherence tool. Device usage information can include data related to a user's device and a mask. Therapy feedback may include data collected during therapy sessions, such as flow data and pressure data. User interactions can involve interactions with a companion application linked to the therapy adherence tool. Alternatively, or additionally, the input data comprises one or more of: a chatlog, pre-identified actions, micro-decisions made by the user, and a topic selected by the user. The user may be categorized into the behavioral subgroup using a k-medoids clustering algorithm or categorized via related outputs from prior subgroup analyses with previous users, such as typing tools based on principal components analyses or other dimension reduction techniques. In some examples, alternative partitioning methods, such as k-means or CLARA, may be used.

A set of initial motivational interviewing questions is generated for the user based on the behavioral subgroup to which the user belongs. User responses to the set of initial motivational interviewing questions are received. A motivational interviewing session is dynamically generated for the user based on the behavioral subgroup and the user response. The motivational interviewing session includes a set of follow-up questions and support messages for the user. The session may deliver tailored cognitive behavioral therapy to the user based on the behavioral subgroup. The generative AI system can adapt the motivational interviewing session to various forms of user input, including written and spoken input. During the motivational interviewing session, behavior change of the user is tracked based on user responses to the set of follow-up questions and support messages. Impact of the tracked behavior change of the user on adherence to the virtual interactive therapy is quantified and used to automatically adapt operation of the PAP therapy device.

For example, when the impact satisfies one or more adjustment criteria, the system generates a device control signal that causes adjustment of one or more operational parameters of the PAP therapy device. The operational parameters may include, without limitation, delivered pressure level, pressure ramp profile, expiratory pressure relief setting, humidification level, mask leak compensation parameters, airflow sensitivity thresholds, or therapy mode selection. In some examples, the adjustment is performed in real time or near real time via a communication interface between the virtual interactive therapy adherence tool and the PAP therapy device. By automatically tuning therapy parameters based on behavior-change-driven adherence impact, the system improves therapy comfort, reduces abandonment risk, and enhances overall therapeutic effectiveness.

In some examples, an AI model associated with the virtual interactive therapy adherence tool is trained based on the impact of the behavior change of the user and the virtual interactive therapy is provided to another user using the trained AI model. In some examples, the virtual interactive therapy is continuous positive airway pressure (CPAP) therapy. The motivational interviewing session is generated for the user who has been diagnosed with sleep apnea such as an obstructive sleep apnea (OSA) or a central sleep apnea (CSA). However, aspects of the disclosure are operable with other therapies.

Examples of the disclosure operate in an unconventional manner at least by integrating a k-medoids clustering algorithm directly into the processor-executed categorization module that processes multimodal input data (e.g., demographic records, real-time PAP-device flow/pressure telemetry, chat logs, spoken utterances, and micro-decision streams) to partition users into latent behavioral subgroups in a single pass, thereby materially reducing the memory footprint and processor cycles required for real-time user typing compared with exhaustive rule-based or brute-force partitioning schemes employed in conventional adherence platforms, and enabling the same hardware resources to support an order-of-magnitude larger concurrent user population without degradation in response latency.

Examples of the disclosure transform a general-purpose computing device into a specialized, behavior-adaptive therapeutic interaction engine that dynamically generates MI dialogues based on real-time user-specific inputs. Unlike static rule-based reminder systems, the disclosed system synthesizes context-aware conversational content using multimodal patient data, thereby improving computational efficiency in generating clinically relevant interventions while reducing redundant processing associated with one-size-fits-all messaging. This technical configuration improves the functioning of the underlying system by enabling fine-grained, data-driven personalization that conventional adherence tools cannot achieve.

Examples of the disclosure further improve computer functionality by implementing a behavioral subgrouping pipeline that applies clustering techniques (e.g., k-medoids) to heterogeneous data streams, including device telemetry, user interaction logs, and demographic attributes. By algorithmically segmenting users into latent behavioral cohorts prior to content generation, the system reduces the search space and computational overhead associated with generating therapeutic interactions, thereby improving processor utilization and memory efficiency. This structured pre-classification enables more deterministic and computing resource-efficient generation of motivational interviewing sequences compared to unguided large language model deployments.

Examples of the disclosure provide a technical improvement in real-time adaptive dialogue generation by coupling generative AI inference with continuous behavior tracking and feedback loops. In particular, the system monitors user responses during an active MI session and dynamically modifies subsequent prompts and support messages without requiring session termination or manual intervention. This closed-loop architecture reduces network round-trips and minimizes latency associated with conventional batch-updated digital therapeutics, thereby improving responsiveness of the virtual therapy interface and enhancing the real-time operational performance of the computing system. Examples of the disclosure introduce a quantification framework that programmatically measures behavior change impact using comparative adherence analytics. By automatically correlating conversational interaction data with therapy usage telemetry, the system executes advanced statistical evaluation routines that continuously retrain and refine the AI model. This self-optimizing pipeline improves model convergence characteristics, reduces manual model-tuning requirements, and enhances long-term predictive accuracy of adherence interventions. As a result, the disclosed system achieves improved computational learning efficiency relative to static digital therapeutic platforms.

Examples of the disclosure further introduce a stateful conversational memory structure that persistently maintains user-specific behavioral context across multiple therapy sessions. The memory structure is indexed using a compact behavioral fingerprint derived from the subgrouping module, enabling O(1)-class retrieval of prior interaction state without requiring full transcript reprocessing. This approach materially reduces processor overhead associated with context reconstruction and enables longitudinal personalization at scale. By improving context retrieval efficiency and reducing repeated natural language processing workloads, the system enhances overall processor throughput and supports sustained multi-session engagement without proportional increases in compute cost.

FIG. 1 is a block diagram illustrating an example generative AI based system 100 to provide virtual interactive therapy for enhanced adherence. A user 114 interacts with a computing system 102 to access a virtual interactive therapy adherence tool 110. The computing system 102 comprises a processor 104 and a memory 106 that stores instructions 108 to access the virtual interactive therapy adherence tool 110 via the network 112. In some examples, the virtual interactive therapy adherence tool 110 is accessed via a browser on the computing system 102. Other ways of accessing the virtual interactive therapy adherence tool 110, such as installing the virtual interactive therapy adherence tool 110 on the computing system 102, are used to implement the system 100 without deviating from the description.

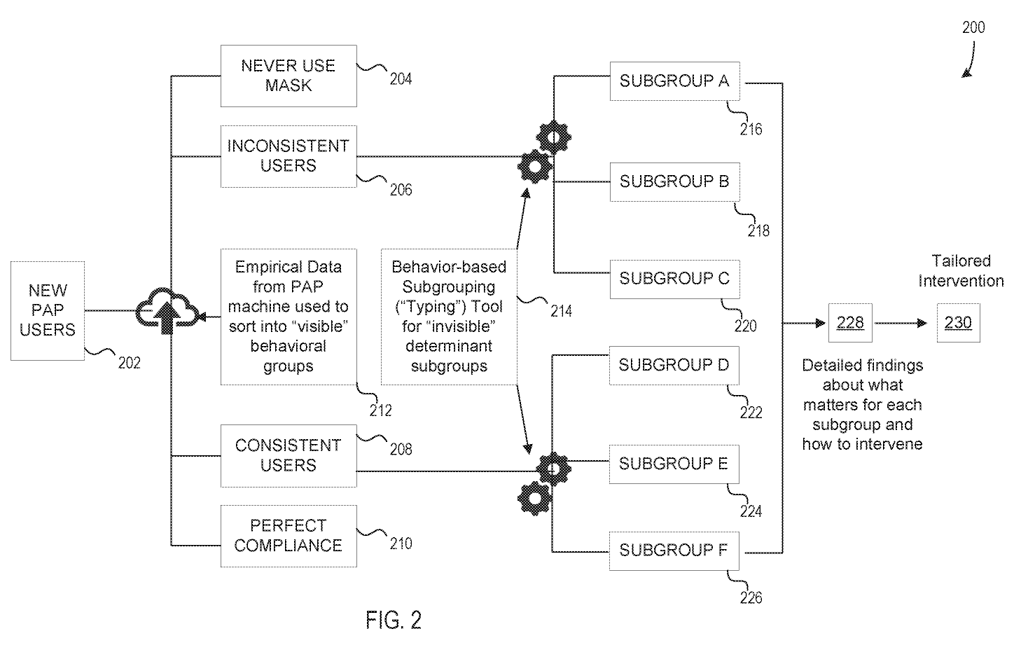

FIG. 2 illustrates an example architecture 200 for combining data from observable and latent variables to provide virtual interactive therapy for adherence. New PAP users 202 are categorized into behavioral subgroups using empirical data from PAP machines 212, leading to tailored interventions. This categorization process is integral to the VITA (Virtual Interactive Therapy for Adherence), which enhances therapy adherence through tailored interventions. The process begins with the identification of new PAP users 202, who are initially sorted into visible groups such as 'Never Use Mask' 204, 'Inconsistent Users' 206, 'Consistent Users' 208, and 'Perfect Compliance' 210. This initial sorting is based on empirical data collected from PAP machines 212, which provides insights into the users' adherence patterns.

A behavior-based subgrouping tool, referred to as ‘typing’ tool 214, is used to categorize users into invisible determinant subgroups labeled A through F (216 through 226). The typing tool 214 leverages highly predictive questions to identify underlying behavioral subgroups that are not immediately visible, with a subjectively high fidelity based on previous clustering analyses. The subgroups correlate with nuanced differences in user behavior, allowing for more precise and effective interventions. Each subgroup is associated with specific findings that lead to the generation of tailored interventions 230 for improving therapy adherence. Detailed findings about each subgroup 228 guide the tailored intervention 230 strategies. These tailored interventions 230 are designed to address the unique needs and challenges of each subgroup (216 through 226), thereby enhancing the overall effectiveness of therapy adherence. The process, illustrated in FIG. 2, emphasizes the importance of understanding user behavior at a granular level to deliver personalized support, which is a key aspect of the VITA approach to improving healthcare outcomes and streamlining healthcare processes. The integration of generative AI technology in this process allows for scalable and adaptive motivational interviewing sessions.

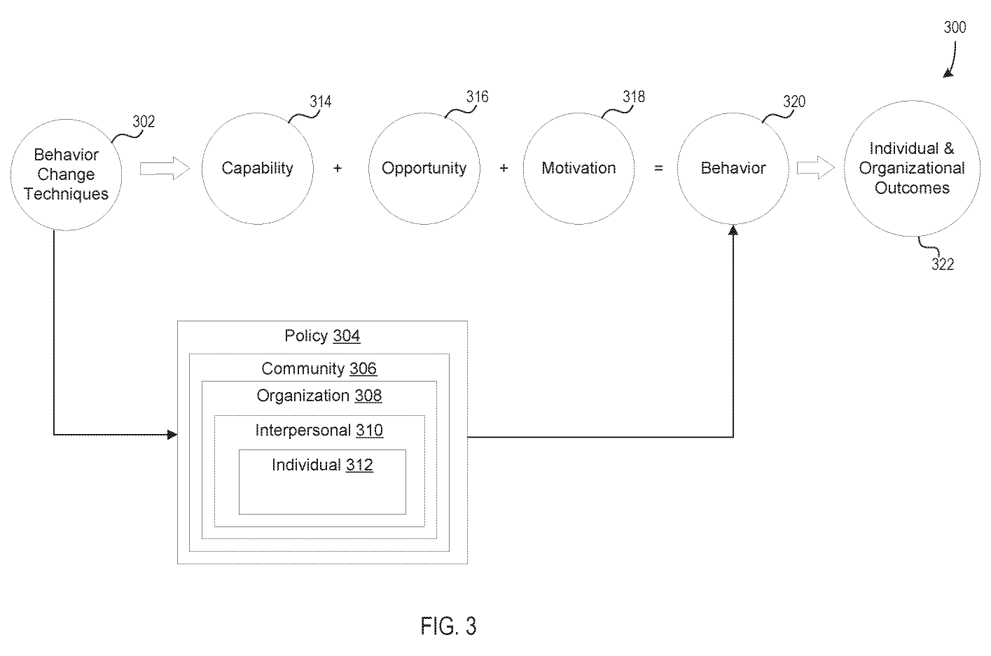

Referring now to FIG. 3, the block diagram 300 visualizes the concept of behavioral determinants and behavior change techniques, which are central to the VITA project's goal of enhancing therapy adherence through motivational interviewing and generative AI. The diagram emphasizes three core domains: capability, opportunity, and motivation, which encompass all modifiable barriers and facilitators that drive behavior change. The behavior change, in turn, results in individual and organizational outcomes, aligning with the VITA project's aim to improve healthcare outcomes and streamline care processes.

FIG. 3 further highlights that behavior change techniques 302 can be implemented at multiple levels, including policy 304, community 306, organization 308, interpersonal 310, and individual 312. The VITA project will affect change in the user through behavior change technique 302 implementation at the individual 312 and interpersonal 310 levels, as appropriate. At the individual level, behavior change techniques 302 include personalized prompts, feedback, goal setting, or self-monitoring features delivered directly to the user. At the interpersonal level, the system employs social support mechanisms, peer comparison, coaching interactions, or collaborative engagement features. As illustrated, these contextual levels are represented as concentric or hierarchically nested domains that collectively shape the environment within which behavior 320 occurs. “Behavior” 320 is functionally determined by the combined effects of Capability 314, Opportunity 316, and Motivation 318, each of which may be influenced by appropriately selected behavior change techniques 302. In this way, the VITA project is designed to use Capability 314, Opportunity 316, and Motivation 318 in a coordinated manner, thereby driving sustained behavior 320 change aligned with the specific individual and organizational outcomes 322.

### Exemplary Implementation

The VITA project aims to enhance therapy adherence through motivational interviewing and generative AI. The focus is on foundational elements such as answering FAQs, receiving feedback, linking to sources, and remembering context so that users can easily find relevant information and maintain continuity in interactions. Speech-to-text capabilities and integration with various data sources enhance the system's ability to support sleep health products and improve data-driven decision-making. Some advanced features such as mouth leak detection, the ability to send and receive images or videos, personalized communications, onboarding support, proactive outreach, and behavioral intervention provide tailored support and interventions, improving user engagement and adherence to therapy. Further, diagnosis interpretation and therapy tracking deliver comprehensive support through motivational interviewing and generative AI. This allows dynamic adaptation to user input and behavior, ultimately enhancing health outcomes and streamlining care processes.

In some examples, speech and text inputs received from the user are processed through a unified multimodal ingestion pipeline that performs automatic speech recognition (ASR), semantic embedding, and confidence scoring prior to downstream behavioral analysis. The pipeline includes noise-robust preprocessing and timestamp alignment to ensure consistency between spoken utterances and device telemetry events. This multimodal synchronization improves accuracy of user-state estimation and reduces error propagation into the behavioral subgrouping and generative dialogue modules.

A Data Input Module may be a component in the system 100 for supporting therapy adherence. This module may be responsible for collecting user data, which may include demographic information, therapy feedback, device usage information, and user interactions with a therapy adherence tool. The data input module may receive input data associated with a user, which may include chat logs, pre-identified actions, micro-decisions, and topics selected by the user. This data may be used to inform the therapy process and enhance user interaction, thereby supporting adherence. The generative AI system may utilize this data to categorize the user into a behavioral subgroup, allowing for tailored interventions. The data input module may also collect data during therapy sessions, such as flow data and pressure data, which may be essential for developing a solution that supports both compliance and long-term adherence. The VITA project leverages this module to enhance patient engagement, improve healthcare outcomes, and streamline the care process. The data input module may work in conjunction with other components, such as the behavioral categorization module and the motivational interviewing module, to ensure a comprehensive approach to therapy adherence. The generative AI system may dynamically generate motivational interviewing sessions adapted to the user based on the behavioral subgroup and user responses, which may include a set of follow-up questions and support messages. The data input module may play a role in ensuring that the system can adapt to various forms of user input, including written and spoken input, thereby enhancing the overall effectiveness of the therapy adherence system.

The Behavioral Categorization Module may categorize users into behavioral subgroups to facilitate tailored interventions. This module may utilize empirical data to determine the appropriate subgroup for each user, which can allow for the generation of initial motivational interviewing questions and actions. The categorization process may involve analyzing input data, which may include demographic information, therapy feedback, and user interactions with therapy adherence tools. The generative AI system may play a role in this process by working out which behavioral subgroup the user belongs to, thereby enabling the creation of personalized interventions. The module may also correlate user responses to initial questions with the behavioral subgroup to dynamically generate a motivational interviewing session. This session may include follow-up questions and support messages, which may be adapted based on the user's responses and subgroup characteristics. The Behavioral Categorization Module may thus serve as a component in the system 100, ensuring that interventions are specifically tailored to the needs and behaviors of individual users, potentially enhancing therapy adherence and improving health outcomes.

The Motivational Interviewing Module may conduct motivational interviewing sessions to support therapy adherence. This module may utilize a generative AI platform to recreate the natural flow of motivational interviewing sessions, adapting to patient input for effective motivational interviewing. The module may generate a set of initial motivational interviewing questions based on the behavioral subgroup of the user. User responses to these questions may be received, allowing the module to dynamically generate a motivational interviewing session tailored to the user. This session may include follow-up questions and support messages, which may be adapted to the user's responses and behavioral subgroup. The module may employ AI-driven motivational interviewing techniques to facilitate self-care discussions and enhance user engagement. The motivational interviewing sessions may be designed to improve adherence by supporting and tracking behavior change. The module may also incorporate motivational enhancement strategies to further improve therapy adherence over time. The generative AI platform may adapt to various forms of patient input, including written and spoken (or audiovisual) input, to ensure the effectiveness of the motivational interviewing process. The integration of these elements may allow the Motivational Interviewing Module to effectively support therapy adherence by enhancing user engagement, improving health outcomes, and streamlining care processes. In some examples, the Motivational Interviewing Module may provide personalized feedback and action plans for patients based on the outcomes of an interviewing session.

A Behavior Tracking Module may be responsible for monitoring user behavior changes during therapy sessions. This module may utilize a generative AI platform to recreate the natural flow of motivational interviewing sessions, which may adapt to patient input for effective motivational interviewing. The module may implement behavior change techniques at various levels, including individual and organizational, to achieve desired outcomes. The generative AI platform may support and track behavior change by adapting to patient input, which may include both written and spoken (or audiovisual) forms. The behavior tracking process may involve monitoring user interactions during motivational interviewing sessions, which may be dynamically generated based on the user's behavioral subgroup and responses. The module may also correlate with the motivational interviewing module to ensure that the motivational interviewing sessions are tailored to the user's needs, thereby enhancing the effectiveness of the therapy adherence system. The behavior tracking module may work in conjunction with other components, such as the data analysis module, to perform statistical analyses on the tracked behavior changes, examining the effectiveness of the therapy adherence system. This comprehensive approach may support the achievement of desired outcomes, such as improved adherence and enhanced user engagement.

The Data Analysis Module may perform statistical analyses to evaluate the effectiveness of the therapy adherence system. This module may be responsible for coupling with advanced statistical analyses to examine the effectiveness of the therapy adherence system. The effectiveness may be assessed by analyzing the tracked behavior changes of users during therapy sessions. The module may generate findings based on these statistical analyses, which may be submitted for peer-reviewed publication. The Data Analysis Module may utilize advanced statistical techniques to ensure that the evaluation of the therapy adherence system is comprehensive and accurate. This module may play a role in validating the impact of the therapy adherence system by providing empirical evidence of its effectiveness. The findings generated by the Data Analysis Module may contribute to the continuous improvement of the therapy adherence system by identifying areas for enhancement. This module may also support the dissemination of research findings to the broader scientific community, thereby contributing to the advancement of knowledge in the field of therapy adherence.

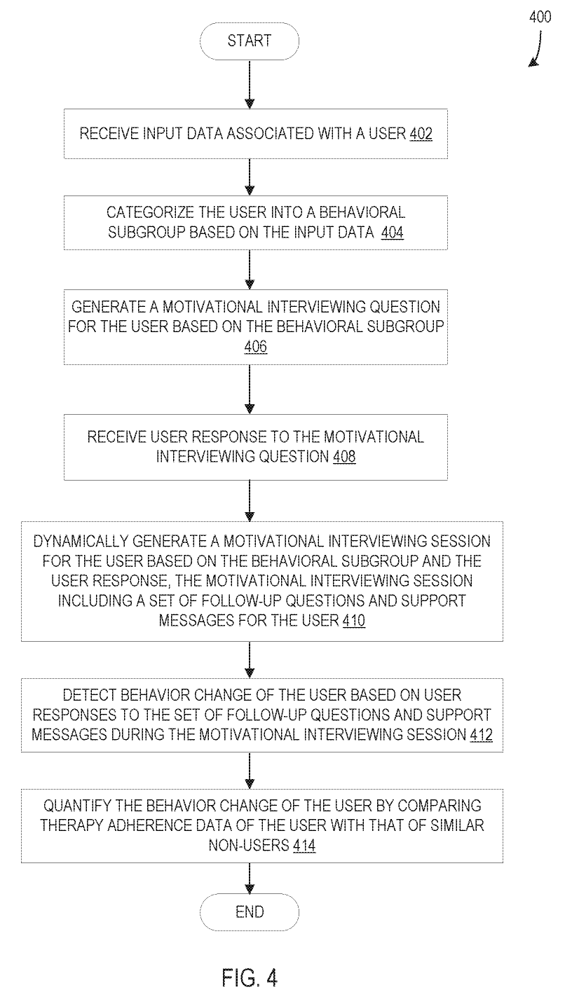

FIG. 4 is a flowchart illustrating an example method 400 for enhancing adherence of a user to a therapy using a generative artificial intelligence (AI) based virtual interactive therapy adherence tool. In some examples, the therapy is prescribed to the user diagnosed with obstructive sleep apnea (OSA) or central sleep apnea (CSA). At 402, input data associated with the user is received. At 404, the user is categorized into a behavioral subgroup based on the input data. At 406, a motivational interviewing question for the user is generated based on the behavioral subgroup. At 408, a user response to the motivational interviewing question is received. At 410, a motivational interviewing session for the user is dynamically generated based on the behavioral subgroup and the user response. The motivational interviewing session includes a set of follow-up questions and support messages for the user. At 412, behavior change of the user is detected based on user responses to the set of follow-up questions and support messages during the motivational interviewing session. At 414, the behavior change of the user is quantified by comparing therapy adherence data of the user (using VITA tool) with that of similar non-users (not using the VITA tool).

In some examples, the quantified impact of the tracked behavior change is converted into a machine-actionable control variable and compared to a stored adjustment threshold retrieved from a memory associated with the system. The system deterministically evaluates whether the quantified impact satisfies the stored adjustment threshold and, responsive to determining that the threshold condition is met, automatically generates a device control signal encoding one or more updated therapy parameter values in accordance with a PAP device communication protocol. The device control signal is transmitted via a wired or wireless communication interface to the PAP therapy device, which authenticates and applies the received control signal to modify one or more operational parameters of the PAP therapy device, such as delivered pressure, ramp profile, expiratory pressure relief setting, humidification level, or airflow sensitivity, thereby enabling closed-loop, behavior-informed adjustment of therapy delivery.

In some examples, an AI model associated with the virtual interactive therapy adherence tool is trained based on the impact of the detected behavior change of the user and the therapy is provided to another user using the trained AI model. This advantageously results in a scalable solution that uses the learnings of behavior change of one user to be applied for enhancing efficacy of the therapy for other users.

In some examples, the categorization of individuals is based on their therapy journey, which is integral to the VITA project's approach to enhancing therapy adherence through tailored interventions. Some categories of individuals are: those beginning their therapy journey, those struggling with adherence to their PAP devices at any point in their journey, and individuals diagnosed with OSA who are hesitant to start therapy or unsure about their ability to adhere. These categories are prioritized to align with the VITA project's goal of enhancing patient engagement and improving healthcare outcomes through motivational interviewing.

In the VITA tool, the individuals who are beginning their therapy journey, may be represented with an icon of a sprouting plant, symbolizing new beginnings and growth. This group may benefit from initial motivational interviewing sessions designed to establish a foundation for adherence. The second category, individuals who are struggling with adherence to their PAP devices at any point in their journey, may be depicted with a concerned face icon, indicating the challenges and uncertainties faced by these individuals. Tailored interventions for this group may focus on addressing specific barriers to adherence and providing ongoing support. The third category, individuals diagnosed with OSA but hesitant to start therapy / unsure about their ability to adhere, may be represented by an icon of a healthcare professional, highlighting the need for professional guidance and reassurance to encourage therapy initiation.

This categorization aligns with the VITA project's use of generative AI to dynamically generate motivational interviewing sessions based on behavioral subgroups. By categorizing individuals in this manner, the VITA project can deliver personalized cognitive behavioral therapy, enhance user engagement, and ultimately improve health outcomes. The integration of these categories into the VITA system allows for a more targeted approach to therapy adherence, leveraging empirical data and user interactions to inform the development of motivational interviewing sessions.

Examples of the disclosure operate in an unconventional and advantageous manner by providing virtual interactive therapy for adherence using a generative AI tool. Further, examples of the disclosure utilize edge large language models (LLMs) that are designed to run on edge devices, such as smartphones, IoT devices, and embedded systems, instead of relying on cloud-based infrastructure. By running locally on edge devices, these models provide lower latency, better privacy, and reduced dependence on constant internet connectivity.

Examples of the disclosure provide a user interface associated with the VITA project, which is designed to support therapy adherence through motivational interviewing. The user interface provides a prompt for an interactive and engaging approach to therapy, encouraging users to actively participate in their treatment process. This aligns with the system's goal of enhancing patient engagement and improving healthcare outcomes through tailored interventions. The user interface may include an option to ‘+’ Add a new module (e.g., a module for Women’s health). This indicates that the user interface is customizable, allowing users to select or add therapy modules that are relevant to their specific needs. This feature supports the system's aim to provide personalized therapy solutions, which can be adapted to various user inputs and preferences. The ability to customize modules aligns with the system's capability to categorize users into behavioral subgroups and generate tailored motivational interviewing sessions based on empirical data and user interactions.

In some examples, AI and machine learning techniques are utilized to train the various modules of the VITA system. In some examples, the machine learning algorithm may use supervised and/or unsupervised techniques, such as those involving artificial neural networks, association rule learning, recurrent neural networks (RNN), Bayesian networks, clustering, deep learning, decision trees, genetic algorithms, Hidden Markov Modeling (HMM), inductive logic programming, learning automata, learning classifier systems, logistic regressions, linear classifiers, quadratic classifiers, reinforcement learning, representation learning, rule-based machine learning, similarity and metric learning, sparse dictionary learning, support vector machines, and/or the like.

Examples of the disclosure ensure secure data management compliant with healthcare regulations like Health Insurance Portability and Accountability Act (HIPAA), General Data Protection Regulation (GDPR), etc. and integrate with existing healthcare systems (such as electronic health record (EHR) or electronic medical record (eMR) systems) to facilitate patient monitoring. This includes encrypted data exchange and storage methodologies to protect sensitive patient information, while also integrating with existing healthcare systems to provide healthcare providers a central dashboard for monitoring patient progress during and after the therapy. VITA may be implemented with a cloud-based architecture to ensure scalability and support accessibility through mobile apps and online platforms, allowing for wide-reaching user support without performance degradation.

VITA incorporates iterative learning mechanisms that continuously update the AI tool based on user interactions and feedback. These iterative updates can involve altering questions and responses to align with user preferences and historical interactions, thereby enhancing the personalization of the therapy experience and potentially leading to greater patient adherence.

### Exemplary Operating Environment

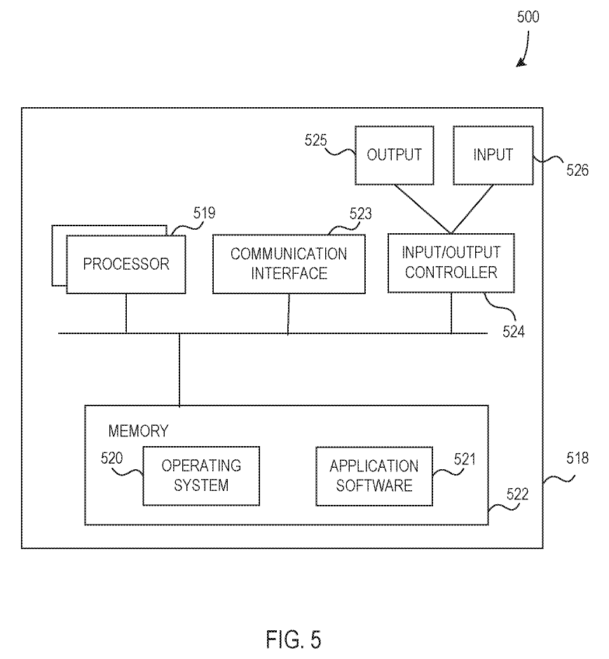

The present disclosure is operable with a computing apparatus according to an embodiment as a functional block diagram 500 in FIG. 5. In an example, components of a computing apparatus 518 are implemented as a part of an electronic device according to one or more embodiments described in this specification. The computing apparatus 518 comprises one or more processors 519 which may be microprocessors, controllers, or any other suitable type of processors for processing computer executable instructions to control the operation of the electronic device. Alternatively, or in addition, the processor 519 is any technology capable of executing logic or instructions, such as a hard-coded machine. In some examples, platform software comprising an operating system 520 or any other suitable platform software is provided on the apparatus 518 to enable application software 521 to be executed on the device. In some examples, implementing the generative AI based tool to provide virtual interactive therapy for adherence is accomplished by software, hardware, and/or firmware.

In some examples, computer executable instructions are provided using any computer-readable media that is accessible by the computing apparatus 518. Computer-readable media include, for example, computer storage media such as a memory 522 and communications media. Computer storage media, such as a memory 522, include volatile and non-volatile, removable, and non-removable media implemented in any method or technology for storage of information such as computer readable instructions, data structures, program modules or the like. Computer storage media include, but are not limited to, Random Access Memory (RAM), Read-Only Memory (ROM), Erasable Programmable Read-Only Memory (EPROM), Electrically Erasable Programmable Read-Only Memory (EEPROM), persistent memory, phase change memory, flash memory or other memory technology, Compact Disk Read-Only Memory (CD-ROM), digital versatile disks (DVD) or other optical storage, magnetic cassettes, magnetic tape, magnetic disk storage, shingled disk storage or other magnetic storage devices, or any other non-transmission medium that can be used to store information for access by a computing apparatus. In contrast, communication media may embody computer readable instructions, data structures, program modules, or the like in a modulated data signal, such as a carrier wave, or other transport mechanism. As defined herein, computer storage media does not include communication media. Therefore, a computer storage medium is not a propagating signal. Propagated signals are not examples of computer storage media. Although the computer storage medium (the memory 522) is shown within the computing apparatus 518, it will be appreciated by a person skilled in the art, that, in some examples, the storage is distributed or located remotely and accessed via a network or other communication link (e.g., using a communication interface 523).

Further, in some examples, the computing apparatus 518 comprises an input/output controller 524 configured to output information to one or more output devices 525, for example a display or a speaker, which are separate from or integral to the electronic device. Additionally, or alternatively, the input/output controller 524 is configured to receive and process an input from one or more input devices 526, for example, a keyboard, a microphone, or a touchpad. In one example, the output device 525 also acts as the input device. An example of such a device is a touch sensitive display. The input/output controller 524 may also output data to devices other than the output device, e.g., a locally connected printing device. In some examples, a user provides input to the input device(s) 526 and/or receives output from the output device(s) 525.

The functionality described herein can be performed, at least in part, by one or more hardware logic components. According to an embodiment, the computing apparatus 518 is configured by the program code when executed by the processor 519 to execute the embodiments of the operations and functionality described. Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Program-specific Standard Products (ASSPs), System-on-a-chip systems (SOCs), Complex Programmable Logic Devices (CPLDs), Graphics Processing Units (GPUs).

At least a portion of the functionality of the various elements in the figures may be performed by other elements in the figures, or an entity (e.g., processor, web service, server, application program, computing device, or the like) not shown in the figures.

Although described in connection with an exemplary computing system environment, examples of the disclosure are capable of implementation with numerous other general purpose or special purpose computing system environments, configurations, or devices.

Examples of well-known computing systems, environments, and/or configurations that are suitable for use with aspects of the disclosure include, but are not limited to, mobile or portable computing devices (e.g., smartphones), personal computers, server computers, hand-held (e.g., tablet) or laptop devices, multiprocessor systems, gaming consoles or controllers, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, and the like. In general, the disclosure is operable with any device with processing capability such that it can execute instructions such as those described herein. Such systems or devices accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions, or the specific components or modules illustrated in the figures and described herein. Other examples of the disclosure include different computer-executable instructions or components having more or less functionality than illustrated and described herein.

In examples involving a general-purpose computer, aspects of the disclosure transform the general-purpose computer into a special-purpose computing device when configured to execute the instructions described herein.

An example generative artificial intelligence (AI) based system provides virtual interactive therapy for enhanced adherence. The system comprises: a processor; and a memory storing instructions that upon execution by the processor cause the processor to: receive input data associated with a user from a positive airway pressure (PAP) therapy device; categorize the user into a behavioral subgroup based on the input data; generate a set of initial motivational interviewing questions for the user based on the behavioral subgroup; receive user response to the set of initial motivational interviewing questions; dynamically generate a motivational interviewing session for the user based on the behavioral subgroup and the user response, the motivational interviewing session including a set of follow-up questions and support messages for the user; track behavior change of the user based on user responses to the set of follow-up questions and support messages during the motivational interviewing session; quantify impact of the tracked behavior change of the user on adherence to the virtual interactive therapy; and based on the impact, cause adjusting one or more operational parameters of the PAP therapy device.

An example computerized method is provided for enhancing adherence of a user to a therapy using a generative artificial intelligence (AI) based virtual interactive therapy adherence tool. The method comprises: receiving input data associated with the user; categorizing the user into a behavioral subgroup based on the input data; generating a motivational interviewing question for the user based on the behavioral subgroup; receiving user response to the motivational interviewing question; dynamically generating a motivational interviewing session for the user based on the behavioral subgroup and the user response, the motivational interviewing session including a set of follow-up questions and support messages for the user; detecting behavior change of the user based on user responses to the set of follow-up questions and support messages during the motivational interviewing session; and measuring impact of the detected behavior change of the user on adherence to the therapy using the generative AI based virtual interactive therapy adherence tool.

An example computer storage medium stores instructions that upon execution by a processor cause the processor to: receive input data associated with a user; categorize the user into a behavioral subgroup based on the input data; generate a set of initial motivational interviewing questions for the user based on the behavioral subgroup; receive user response to the set of initial motivational interviewing questions; dynamically generate a motivational interviewing session for the user based on the behavioral subgroup and the user response, the motivational interviewing session including a set of follow-up questions and support messages for the user; track behavior change of the user based on user responses to the set of follow-up questions and support messages during the motivational interviewing session; and quantify impact of the tracked behavior change of the user on adherence to the virtual interactive therapy.

Alternatively, or in addition to the other examples described herein, examples include any combination of the following:

wherein the input data comprises one or more of: demographic information, therapy feedback from the user, device usage information, and user interactions with a therapy adherence tool.

wherein the input data further comprises one or more of: a chatlog, pre-identified actions, micro-decisions made by the user, and a topic selected by the user.

wherein the user is categorized into the behavioral subgroup using k-medoids clustering algorithm.

wherein the virtual interactive therapy is continuous positive airway pressure (CPAP) therapy;

wherein the motivational interviewing session is generated for the user diagnosed with obstructive sleep apnea (OSA) or central sleep apnea (CSA).

wherein the therapy is prescribed to the user diagnosed with obstructive sleep apnea (OSA) or central sleep apnea (CSA).

training an AI model associated with the virtual interactive therapy adherence tool based on the impact of the detected behavior change of the user; and providing the therapy to another user using the trained AI model.

Any range or device value given herein may be extended or altered without losing the effect sought, as will be apparent to the skilled person.

Examples have been described with reference to data monitored and/or collected from the users (e.g., user identity data with respect to profiles). In some examples, notice is provided to the users of the collection of the data (e.g., via a dialog box or preference setting) and users are given the opportunity to give or deny consent for the monitoring and/or collection. The consent takes the form of opt-in consent or opt-out consent.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items.

The embodiments illustrated and described herein as well as embodiments not specifically described herein but within the scope of aspects of the claims constitute an exemplary means for providing virtual interactive therapy for adherence.

The term “comprising” is used in this specification to mean including the feature(s) or act(s) followed thereafter, without excluding the presence of one or more additional features or acts.

In some examples, the operations illustrated in the figures are implemented as software instructions encoded on a computer readable medium, in hardware programmed or designed to perform the operations, or both. For example, aspects of the disclosure are implemented as a system on a chip or other circuitry including a plurality of interconnected, electrically conductive elements.

The order of execution or performance of the operations in examples of the disclosure illustrated and described herein is not essential, unless otherwise specified. That is, the operations may be performed in any order, unless otherwise specified, and examples of the disclosure may include additional or fewer operations than those disclosed herein. For example, it is contemplated that executing or performing a particular operation before, contemporaneously with, or after another operation is within the scope of aspects of the disclosure.

When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A generative artificial intelligence (AI) based system to provide virtual interactive therapy for enhanced adherence, the system comprising:
a processor; and
a memory storing instructions that upon execution by the processor cause the processor to:
receive input data associated with a user from a positive airway pressure (PAP) therapy device;
categorize the user into a behavioral subgroup based on the input data;
generate initial motivational interviewing questions for the user based on the behavioral subgroup;
receive user responses to the initial motivational interviewing questions;
dynamically generate a motivational interviewing session for the user based on the behavioral subgroup and the user responses, the motivational interviewing session including follow-up questions and support messages for the user;
track a behavior change of the user based on user responses to the follow-up questions and support messages during the motivational interviewing session;
quantify impact of the tracked behavior change of the user on adherence to the virtual interactive therapy; and
based on the impact, cause one or more operational parameters of the PAP therapy device to be adjusted.

2. The system of claim 1, wherein the input data comprises one or more of: demographic information, therapy feedback from the user, device usage information, and user interactions with a therapy adherence tool.

3. The system of claim 2, wherein the input data further comprises one or more of: a chatlog, pre-identified actions, micro-decisions made by the user, and a topic selected by the user.

4. The system of claim 1, wherein the user is categorized into the behavioral subgroup using k-medoids clustering algorithm.

5. The system of claim 1, wherein the virtual interactive therapy is continuous positive airway pressure (CPAP) therapy.

6. The system of claim 1, wherein the motivational interviewing session is generated for the user diagnosed with obstructive sleep apnea (OSA) or central sleep apnea (CSA).

7. A computerized method for enhancing adherence of a user to a therapy using a generative artificial intelligence (AI) based virtual interactive therapy adherence tool, the method comprising:
receiving input data associated with the user;
categorizing the user into a behavioral subgroup based on the input data;
generating a motivational interviewing question for the user based on the behavioral subgroup;
receiving a user response to the motivational interviewing question;
dynamically generating a motivational interviewing session for the user based on the behavioral subgroup and the user response, the motivational interviewing session including a follow-up question and support message for the user;
detecting a behavior change of the user based on a user response to the follow-up question and support message during the motivational interviewing session; and
measuring impact of the detected behavior change of the user on adherence to the therapy using the generative AI based virtual interactive therapy adherence tool.

8. The method of claim 7, wherein the therapy is prescribed to the user diagnosed with obstructive sleep apnea (OSA) or central sleep apnea (CSA).

9. The method of claim 7, further comprising:
training an AI model associated with the virtual interactive therapy adherence tool based on the impact of the detected behavior change of the user; and
providing the therapy to another user using the trained AI model.

10. The method of claim 7, wherein the input data comprises one or more of: demographic information, therapy feedback from the user, device usage information, and user interactions with a therapy adherence tool.

11. The method of claim 10, wherein the input data further comprises one or more of: a chatlog, pre-identified actions, micro-decisions made by the user, and a topic selected by the user.

12. The method of claim 7, wherein the user is categorized into the behavioral subgroup using k-medoids clustering algorithm.

13. The method of claim 7, wherein the AI based virtual interactive therapy adherence tool provides continuous positive airway pressure (CPAP) therapy.

14. A computer storage medium storing instructions to provide virtual interactive therapy for enhanced adherence, the instructions upon execution by a processor cause the processor to:
receive input data associated with a user;
categorize the user into a behavioral subgroup based on the input data;
generate a set of initial motivational interviewing questions for the user based on the behavioral subgroup;
receive user response to the set of initial motivational interviewing questions;
dynamically generate a motivational interviewing session for the user based on the behavioral subgroup and the user response, the motivational interviewing session including a set of follow-up questions and support messages for the user;
track behavior change of the user based on user responses to the set of follow-up questions and support messages during the motivational interviewing session; and
quantify impact of the tracked behavior change of the user on adherence to the virtual interactive therapy.

15. The computer storage medium of claim 14, wherein the input data comprises one or more of: demographic information, therapy feedback from the user, device usage information, and user interactions with a therapy adherence tool.

16. The computer storage medium of claim 15, wherein the input data further comprises one or more of: a chatlog, pre-identified actions, micro-decisions made by the user, and a topic selected by the user.

17. The computer storage medium of claim 14, wherein the user is categorized into the behavioral subgroup using k-medoids clustering algorithm.

18. The computer storage medium of claim 14, wherein the virtual interactive therapy is continuous positive airway pressure (CPAP) therapy.

19. The computer storage medium of claim 14, wherein the motivational interviewing session is generated for the user diagnosed with obstructive sleep apnea (OSA) or central sleep apnea (CSA).

20. The computer storage medium of claim 14, wherein the instructions upon execution by the processor further cause the processor to:
train an AI model associated with a virtual interactive therapy adherence tool based on the impact of the behavior change of the user; and
provide the virtual interactive therapy to another user using the trained AI model.
